(19) United States
(12) Patent Application Publication
(10) Pub. No.: US 2009/0163526 A1
(43) Pub. Date: Jun. 25, 2009

(54) Process for the Preparation of an Anti Microbial Extract from Leaves of the Plant Callistemon Rigidus

(75) Inventor: Sanjai Saxena, Punjab (IN)

Correspondence Address: THE WEBB LAW FIRM, P.C., 700 KOPPERS BUILDING, 436 SEVENTH AVENUE, PITTSBURGH, PA, 15219, US

(73) Assignees: DEPARTMENT OF BIOTECHNOLOGY, New Delhi (IN); THAPAR INSTITUTE OF ENGINEERING & TECHNOLOGY, Patiala (IN)

(21) Appl. No.: 11/995,412

(22) PCT Filed: Mar. 22, 2006
(86) PCT No.: PCT/IN06/00071

§ 371 Date: Jul. 30, 2008

(30) Foreign Application Priority Data
Jul. 11, 2005 (IN) 1795/DEL/2005

(51) Int. Cl. A61K 31/435; B01D 15/24

(52) U.S. Cl. 514/277; 210/635

(57) ABSTRACT

A process for the preparation of an antimicrobial extract from leaves of the plant Callistemon rigidus by cold extract using organic solvents selected from methanol, ethanol, chloroform, dichlorom ethane, hexane, diethyl and ether, concentrating the filtrate in vacuo to obtain the crude extract.

### FIELD OF INVENTION

This invention relates the extracts of the plant Callistemon rigidus R. Br. having antimicrobial and/or antibiotic activity and a process for preparation of an anti-infective leaf extract to combat multidrug resistant infectious microorganisms.

### PRIOR ART

It is generally accepted that drug resistant microorganisms is higher compared to the pace of discovery of new drugs. Formulations having a combination of two drugs have failed in overcoming the problem of drug resistance since these are old compounds resistance against which has already been acquired by the pathogens. Derivatives of these drugs provide a temporary solution before the microorganism also develops drug resistance. The drug resistant microorganisms are responsible for a variety of chronic infections and their treatment demands the use of more expensive and toxic drugs.

Plants are being screened for their pharmacological activities including antimicrobial and immunomodulatory activities for bioassay-guided isolation of the bioactive compounds or lead fractions. These bioactive compounds can be used directly or serve as templates for development of superior versions of compounds inhibiting multidrug resistant infectious microorganisms. The lead fractions can be used in the development of standardized herbal formulation in the form of herbal extract/syrup/tablet (for oral use), topical lotion or a cream.

Callistemon rigidus R.Br. (Family: Myrtaceae), commonly known as bottle brush is a native of Australia and naturalized in India. The plants is used as a bush food by aboriginals in Australia and the decoction of leaves implicated for cure against cough, bronchitis and Respiratory tract infections (RTI's) (Jirovetz et.al., 1997). There is only a single record on the phytochemical analysis of dried leaves of Callistemon rigidus R.Br by head space gas chromatography (Jirovetz et.al., 1998) and there exists no documental evidence of its pharmaceutical (antimicrobial) activity to date.

A variety of plant extracts or their fractions compound have been developed as standardized formulations for use as bioenhancers of drug action (United States Patent Application 200301703236, G. N. Qazi et. al. United States Patent Application 20030228381, G. N. Qazi et al.) in the treatment of various diseases like Arthritis, Cardiovascular diseases etc. There are only a few polyherbal compositions (a mixture of two or more plant extracts), which have an antibacterial action and are solely used for treating infectious diseases (U.S. Pat. No. 5,834,000, 1998 Yng_Wong). These polyherbals formulations are generally oriented towards superficial skin infections and not systemic diseases (U.S. Pat. No. 6,365,197, 2002, Y N Shukla et. al.; U.S. Pat. No. 5,840,310, 1997, Wambebe et. al.;) or the spectrum of activity if a single plant is used is narrow either gram positive or gram negative (U.S. Pat. No. 6,316,033, 2001, Yuan Lin; U.S. Pat. No. 5,560,912, 1996, I Neeman et. al)

The major drawbacks has been that the anti-infective herbal formulations so far developed are polyherbals-containing more than one plant extracts so their standardization takes a very long time, moreover the formulations so far developed have been oriented primarily towards bacterial and fungal infections of skin. There are only a few formulations, which work against bacterial systemic infections. If a standardized phytopharmaceutical formulation is developed then the spectrum of its activity is narrow. These formulations generally do not work against infectious diseases caused by MDR pathogens.

### OBJECTS OF THE INVENTION

An object of this invention is to propose an organic leaf extract of Callistemon rigidus R.Br. having significant antimicrobial activity against multidrug resistant microorganisms.

Yet another object of this invention is to propose a process of isolation of lead fractions of the organic leaf extract of Callistemon rigidus R.Br. responsible for an antimicrobial action.

A further object of this invention is to propose a process for preparation of organic leaf extract of Callistemon rigidus R.Br. having significant antimicrobial activity against multidrug resistant microorganisms over standard antibiotic against drug resistant microorganisms.

Still another object of the present invention is to propose a safe, cost effective herbal formulation(s) having broad-spectrum activity against both gram-positive and gram-negative drug resistant microorganisms and better activity when compared to individual leads or commercial antibiotics under in vitro conditions.

### DESCRIPTION OF INVENTION

According to this invention there is provided a process for the preparation of an antimicrobial extract from leaves of the plant Callistemon rigidus by cold extraction using organic solvents selected from methanol, ethanol, chloroform, dichloromethane, hexane, diethyl and ether, concentrating the filtrate in vacuo to obtain the crude extract.

The step of solvent extraction is carried out at a temperature of 15 to 25° C. for 48 to 96 hours.

The crude extract is subjected to the step of column chromatography, a step gradient being used as the mobile phase in a silica gel column packed in a non polar solvent, such as chloroform, ether or carbon tetrachloride to obtain fractions, subjecting the fractions to the step of evaporation and purification. The step of purification comprises in using silica gel packed in a polar solvent such as methanol, a separation being carried out using mobile phase of organic solvents such as chloroform, methanol, ethyl acetate and ammonia followed by a different concentration of acetone, chloroform, methanol and finally by acetone, methanol, acetone, methanol, acetone, water to obtain four fractions of the crude extract.

The invention further comprises a herbal composition comprising said four fractions in sub-inhibitory concentrations ranging between MIC and 0.125 MIC and having a potential in vitro antibacterial activity with 5-7 log reduction in the colony counts of different test organisms with no post antibiotic effect when compared to Cefuroxime sodium and having a FIC index of 0.167

The first fraction has a MIC of 0.39 μg/ml for S.aureus (UTI isolate), a MIC of 3.125 μg/ml for S.aureus (UTI isolate 2); S.aureus (food isolate 1), E.coli O157:H7 (isolate 2), E.coli (UTI isolate) and E.coli (GI isolate), a MIC of 1.56 μg/ml for E.coli NCTC 10418 and E.coli O157:H7 (isolate1)

The second fraction has a MIC of 3.125 μg/ml for S.aureus (UTI isolate 1), E.coli 0157:H7 and E.coli (UTI isolate), a MIC of 1.56 μg/ml for S.aureus (UTI isolate 1), E.coli NCTC 10418 and E.coli (GI isolate), a MIC of 12.5 μg/ml for S.aureus (Food isolate 1) and S. aureus (Food isolate 2) and 25 μg/ml for E.coli O157:H7 (isolate 2)

The third fraction has a MIC of 0.78 μg/ml against S.aureus (UTI isolate 1), MIC of 3.125 μg/ml for S.aureus (UTI isolate 2), S. aureus (Food isolate 1), E.coli 0157:H7 (isolate 1) and E.coli (UTI isolate)

The fourth fraction has a MIC of 6.25 μg/ml to 400 μg/ml against the test organisms.

### 1) EXAMPLE

Fresh plant material (leaves) of Callistemon rigidus were collected from Thapar Technology Campus (TTC), washed thoroughly and air-dried. These were then ground into a paste, weighed and subjected to the process of extraction using solvents like methanol, acetone, chloroform and ethyl acetate under cold condition (15-25° C.) for 48-96 hours. The filtrate of each solvent was obtained using filter paper Whatmann no. 1 and was concentrated in vacuo to obtain residues. These residues so obtained were tested for their antimicrobial activity against clinical isolates and control isolates of Staphylococcus aureus, Staphylococcus epidermidis, Escherichia coli, E.coli O157;H7, Pseudomonas aeruginosa and Candida albicans by agar well diffusion assay (Lehrer, 1991). The control organisms were NCTC strains, Staphylococcus aurens NCTC 6571,

E. Coli NCTC 10418, P. aeruginosa NCTC 10668 and Candida albicans. Based on these results the Minimal Inhibitory Concentration (MIC) of the raw leaf extracts against the test microorganisms were determined against the test microorganisms by Microbroth dilution method (EUCAST, 2000).

Column chromatography was used for the separation of the crude extract. A step gradient of Chloroform: Dichloromethane: Acetone: Ethyl acetate was used as the mobile phase in a silica gel column (60-120 μm mesh size) packed in a non-polar solvent like chloroform. Ether or Carbon tetrachloride. All the fractions so obtained were evaporated at 40° C. in a flash evaporator. Further fractionation and confirmation of the antibacterial activity was ascertained by direct bioautography wherein the column fractions were spotted over 20×20 cm Merck Si-Gel60. F₂₅₄ 0.25 mm thick plates and developed with a mobile phase comprising of different concentrations of Ethyl acetate: Chloroform: Methanol: Ammonia. The spots and bands were visualized under UV (254 and 366 nm) and Visible light and their R_{f} (retention frequency) values were recorded. These were recovered from the TLC plates and clubbed together on the basis of their R_{f} values and analyzed spectroscopically to confirm their purity and determination of their λ_{max.} The concentration used for spectroscopic determination was 5 mg/ml and wavelength scan was carried out in the range of 190-840 nm.

Mass purification of the crude herbal extract was then carried out in a 50 cm×25 mm glass column using silica gel (60-120 μm mesh size) packed in a polar solvent like methanol. The separation was done using mobile phase comprising of a defined concentration of organic solvents like Chloroform; Methanol, Ethyl Acetate and Ammonia followed by different concentrations of Acetone; Chloroform; Methanol, then by Acetone, Methanol; Acetone; Methanol Acetone; Water ad finally the column was washed with water. The antibacterial activity of the column fractions was ascertained by Agar Well diffusion assay. Microbroth dilution method was used to determine the MIC of antimicrobial fractions obtained after mass purification by column chromatography.

A standardized formulation (herbal composition) was designed using these purified leads by using the most potent and the least potent in different concentrations ranging from ⅛ trh of MIC to 6 times of MIC using 96-well microtiter plate by checkerboard method (Climo, 2001). The formulation exhibiting maximum inhibition was again tested in different concentration ranging from ⅛^{th} of MIC to 6 times of MIC was combined with other fraction in a similar way to design a standardized anti-infective broad spectrum herbal formulation. The in vitro antimicrobial activity of the herbal formulations with positive control i.e. Cefuroxime sodium was established.

Callistemon rigidus R.Br. has not been used as a therapeutic agent so far either singly or in combination with other medicinal plants. The lead extracts exhibit a broad spectrum of antibacterial activity inhibiting both gram positive and gram-negative microbes. The lead fractions also exhibit a potential broad-spectrum antibacterial activity against the test microbes. The lead fractions as well as the crude extract are active against microbes causing systemic infections, food borne infections as well as skin infections. The herbal formulations developed are also active against microbes causing systemic infections, food borne infections as well as skin infections.

The MIC of these lead fractions is much lower in a range of 0.39 μg/ml-12.5 μg/ml as compared to 12.5 μg/ml-400 μg/ml for Cefuroxime sodium in drug resistant infectious microorganisms indicating a very potential antimicrobial action in comparison to most potent antibiotics in pharmacopoeia.

A novel process for preparation of an antimicrobial extract wherein the fresh leaves of the plant Callistemon rigidus are used by the process of cold extraction using organic solvents like methanol, ethanol, chloroform, dichloromethane, hexane, diethyl, ether under defined temperature for a period of 2-3 days for evaluation of the bioactivity.

“First documental evidence” of the “broad-spectrum antimicrobial activity” of organic extracts of leaves of Callistemon rigidus as per claim 1 indicating its possible pharmacological use as an Anti-infective agent.

Total organic solvent extract hereinafter referred as “Crude extract” from the leaves of Callistemon rigidus exhibited the maximum inhibition and broadest spectrum of antimicrobial activity in agar well diffusion assay when compared to Cefdinir (a third generation cephalosporin).

The crude extract residue from Callistemon rigidus leaves as per the method in claim 1 has a higher antimicrobial activity as well as yield as compared to one obtained by hot solvent extraction using a soxhlet extractor. Crude extract had a MIC of 450 μg/ml against Staphylococcus aureus NCTC 6571, Staphylococcus aureus (Urinary tract infection) isolate=1, Staphylococcus aureus (Urinary Tract Infection) isolate-2, Staphylococcus aureus (food isolate) and Staphylococcus epidermidis (Clinical isolate). Crude extract had a MIC of 300 μg/ml against Escherichia coli (urinary tract infection) isolate, E. coli NCTC 10418, E. coli O157;H7. The crude extract had a MIC of 2700 μg/ml against Pseudomonas aeruginosa NCTC 10668, Pseudomonas aeruginosa (Urinary tract isolate) and MIC of 1626 μg/ml against Candida albicans. The crude extract at the MIC concentration against respective organisms induced a 3-log reduction (99% Kill) within 6-9 hours under in vitro conditions.

TLC fractionation of the column fraction employed a defined concentration of ethyl acetate; chloroform: Methanol: ammonia as a mobile phase. The Rf values of the fraction ranged from 0.17 to 1.00. Mass purification of the crude extract was effected with a defined concentration of ethyl acetate: Chloroform: Methanol: Ammonia as a mobile phase gave 10 fractions and their spectral range was between 264-531 nm. Only four column fractions viz. F1, F3, F4 and F4b exhibited the antimicrobial activity by agar well diffusion assay.

1) A process for the preparation of an antimicrobial extract from leaves of the plant Callistemon rigidus by cold extract using organic solvents selected from methanol, ethanol, chloroform, dichloromethane, hexane, diethyl and ether between 15 to 25° C. at 120 rpm for 38-96 hrs, concentrating the filtrate in vacuo to obtain the crude extract

2) A process as claimed in claim 1 wherein the step of solvent extraction is carried out preferably at a temperature of 25° C.

3) A process as claimed in claim 1 wherein the crude extract is subjected to the step of column chromatography, a step gradient being used as the mobile phase in a silica gel column packed in a non polar solvent, such as chloroform, ether or carbon tetrachloride to obtain fractions, subjecting the fractions to the step of evaporation and purification.

4) A process as claimed in claim 3 wherein the step of purification comprises in using silica gel packed in a polar solvent such as methanol, a separation being carried out using mobile phase of organic solvents such as chloroform, methanol, ethyl acetate and ammonia followed by a different concentration of acetone, chloroform, methanol and finally by acetone, methanol, acetone, methanol, acetone, water to obtain four fractions of the crude extract.

5) A herbal composition comprising a mixture of four alkaloids having Rf 0.86, 0.99, 0.7. 0.6 obtained through TLC for fraction 1, 2, 3 & 4 wherein said four fractions as claimed in claim 4 in sub-inhibitory concentrations ranging between MIC and 0.125 MIC and having a potential in vitro antibacterial activity with 5-7 log reduction in the colony counts of different test organisms with no post antibiotic effect when compared to Cefuroxime sodium and having a FIC index of 0.167

6) A herbal composition as claimed in claim 5 wherein the first fraction has a MIC of 0.39 μg/ml for S.aureus (UTI isolate), a MIC of 3.125 μg/ml for S.aureus (UTI isolate 2); S.aureus (food isolate 1), E.coli O157:H7 (isolate 2), E.coli (UTI isolate) and E.coli (GI isolate), a MIC of 1.56 μg/ml for E.coli NCTC 10418 and E.coli O157:H7 (isolate 1)

7) A herbal composition as claimed in claim 5 wherein the second fraction has a MIC of 3.125 μg/ml for S.aureus (UTI isolate 1), E.coli 0157:H7 and E.coli (UTI isolate), a MIC of 1.56 μg/ml for S.aureus (UTI isolate 1), E.coli NCTC 10418 and E.coli (GI isolate), a MIC of 12.5 μg/ml for S.aureus (Food isolate 1) and S. aureus (Food isolate 2) and 25 μg/ml for E.coli O157:H7 (isolate 2)

8) A herbal composition as claimed in claim 5 wherein the third fraction has a MIC of 0.78 μg/ml against S.aureus (UTI isolate 1), MIC of 3.125 μg/ml for S.aureus (UTI isolate 2), S. aureus (Food isolate 1), E.coli 0157:H7 (isolate 1) and E.coli (UTI isolate)

9) A herbal composition as claimed in claim 5 wherein the third fraction has a MIC of 6.25 μg/ml to 400 μg/ml against the test organisms.
